(19) United States
(12) Patent Application Publication
(10) Pub. No.: US 2017/0241980 A1
(43) Pub. Date: Aug. 24, 2017

(54) Assay for FGF2 Secretion and Signaling

(71) Applicant: Universitaet Heidelberg, Heidelberg (DE)

(72) Inventor: Walter Nickel, Schriesheim (DE)

(21) Appl. No.: 15/510,177

(22) PCT Filed: Oct. 12, 2015
(86) PCT No.: PCT/EP2015/073591

§ 371 Date: Mar. 9, 2017

(30) Foreign Application Priority Data
Oct. 10, 2014 (GB) 1417961.8

(51) Int. Cl. G01N 33/50; G01N 33/74

(52) U.S. Cl. CPC G01N 33/5011; G01N 33/74; G01N 2333/50; G01N 2500/10

(57) ABSTRACT

A method for the identification of compounds modulating the biological activity of FGF2, the method comprising the use of target cells sensitive to the presence of FGF2 by obstructing proliferation, wherein the target cells constitutively express a first reporter in the nucleus of living target cells.

### FIELD OF THE INVENTION

The present invention relates to a method for the identification of FGF2 secretion and/or signalling and use of the method.

### BACKGROUND OF THE INVENTION

Growth factors belong to a group of proteins that stimulate the growth of specific tissues. Fibroblast growth factors (FGFs) are small polypeptide growth factors and belong to the family of growth factors. They share certain structural characteristics and most of them bind to heparin. Many FGFs contain signal peptides for secretion and are secreted into the extracellular matrix (EM). FGFs bind specific receptors and induce by their binding the activation of intracellular signal cascades.

Epithelial and tumor cells for instance produce FGF2 or basic FGF, which does not contain a signal sequence for secretion. A nuclear localization sequence has been reported upstream of the AUG start codon (Bugler et al., 1991, MCB 11, 4770-4777), although the role of nuclear localization remains unclear.

Ramasamy and colleagues (Blood, 2010, Vol. 116, No. 21, p. 432; British Journal of Hematology, 2012, 157, p. 564) describe an in-vitro experimental platform for high throughput analysis of the effect on multiple myeloma cells and the tumor microenvironment in a co-culture setting. They generated eGFP expressing multiple myeloma (MM) cells and other cancer derived cell lines expressing eGFP. The use of eGFP-MM cells allows flow cytometry analysis of cell cycle profile and apoptosis with no significant cellular contamination from co-cultured cells such as fibroblasts, osteoclasts or stromal cells derived from bone marrow aspirates of MM patients. Using cancer derived cell lines, the platform of Ramasamy and colleagues is not appropriate for detecting FGF2.

Flaberg an colleagues (Int. Journal of Cancer, 2011, Vol. 128, No. 21, p. 2793) teach a highthrouput system employing a lymph node metastasis derived cell line that is stably transfected with a constitutive CMV promotor driven eGFP. Other cancer derived cell lines were transfected with recombinant histone H2A-red fluorescent protein. However, the disclosed system is based on cancer cell line and for that reason not suitable for detecting FGF2 secretion.

Fuijata et al (Cancer Science, 2009, Vol. 100, No. 12, p. 2390) disclose a simplified direct co-culture system that is able to quantify populations of cancer cells in co-culture. They established three eGFP expressing pancreatic cancer cell lines and were able to quantify them reliable and reproducible whenever co-cultured with activated pancreatic stellate cells.

In particular, tumor cells express FGF2. Thus, inhibitors of FGF2 secretion and/or signaling seem to be potential targets for tumor therapy. Thus, there is a need for an assay allowing for instance high-throughput screens of compound libraries for effective inhibitors of FGF2. There is a need for an assay being capable of measuring a physiological response to the presence of FGF2.

### BRIEF DESCRIPTION OF THE INVENTION

The present disclosure provides a method for the identification of FGF2 modulating compounds, the method comprising the use of target cells sensitive to the presence of FGF2 by obstructing proliferation, wherein the target cells constitutively express a first reporter in the nucleus of living target cells. The first reporter can be a first fluorescent protein.

A second reporter may further be used for detecting apoptotic cells and the second reporter may be a second fluorescent protein.

The absorption spectrum of the first and second fluorescent protein may differ so that they can even be distinguished using a microscope.

It is intended that the fluorescence of first and/or second reporter will be determined and may form the basis for calculating the quotient of the fluorescence of the first and second reporter.

The target cells may be co-incubated with cells or a liquid to detect the secretion or presence of FGF2 or compounds effecting FGF2 signaling.

The target cells may further be co-incubated with FGF2 secreting cells and the FGF2 secretion in the FGF2 secreting cells may be inducible.

The FGF2 secreting cells can be coincubated with at least one compound selected from the group comprising peptides, proteins, nucleic acids, carbohydrates, antibodies, lipids, micelles, vesicles, synthetic molecules and polymers.

The number of living target cells may be monitored by determining fluorescence of the first reporter.

The FGF2 secreting cells may be selected from the group comprising tumor cells, endothelial cells, muscle cells, neural cells and fibroblasts.

It is further intended that additionally control cells may be used, which are constitutively expressing a third reporter, wherein the control cells are insensitive to the presence of FGF2.

The target cells may be genetically modified cells derived from a neuroblastoma, wherein the target cells may be SK-N-MC cells.

It is further envisaged that at least one compound library can be used for co-incubation with the target cells, with compounds being bound to at least one of metal particles, nanoparticles, or a solid phase and the compounds being selected from the group comprising peptides, proteins, carbohydrates, antibodies, lipids, micelles, vesicles, synthetic or biological molecules and polymers.

The method may further employ at least one compound library that may be used for co-incubation with the target cells, wherein the compounds of the library are in solution.

The target cells may be cultivated in multiwell plates with each well comprising different compounds to be tested for their ability to modulate FGF2 secretion and/or signalling.

The FGF2 secreting cells may be separated from target cells by cell sorting, such as fluorescence activated cell sorting or magnetic cell sorting, and re-cultured for further approaches.

Another object of the instant disclosure is a use of the method as disclosed above for distinction between FGF2 secretion and FGF2 signaling modulating compounds, high-throughput screens, diagnosis of tumor-associated diseases, monitoring of cell proliferation, identification of compounds for treatment and/or prevention of tumor-associated diseases.

Further, a kit for the identification of FGF2 modulating compounds is an object of the instant disclosure, the kit comprising target cells constitutively expressing a first reporter in the nucleus having a first fluorescence, wherein the target cells are sensitive to the presence of FGF2.

Such a kit may comprise a second reporter having a second fluorescence with a different absorption spectrum from the first fluorescence of the first reporter, wherein the second reporter detects apoptotic cells.

The kit may further comprises control cells constitutively expressing a third reporter having a third fluorescence different from the first and second fluorescence of first and second reporter, wherein the control cells are insensitive to the presence of FGF2.

Such a kit may comprises recombinant FGF2 and further FGF2 secreting cells, wherein wih FGF2 secretion may be inducible.

### BRIEF DESCRIPTION OF THE FIGURES

FIG. 1 Proliferation of target cells expressing constitutively a fluorescent protein in the presence and absence of FGF2.

FIG. 2 Proliferation of target cells expressing constitutively a fluorescent reporter co-incubated with HeLa cells secreting FGF2 inducible with doxycycline.

FIG. 3/4 Effect of different FGF2 concentrations on SK-N-MC cell proliferation

FIG. 5/6 Effect of different secreted FGF2 concentrations on SK-N-MC cells co-incubated with HeLa cells secreting FGF2 inducible with doxycycline.

FIG. 7 Quotient of red fluorescent nuclei and green fluorescence caused by apoptotic cells in the absence and presence of FGF2.

FIG. 8 Quotient of red fluorescent nuclei and green fluorescence caused by apoptotic cells in the absence and presence of doxycycline induced FGF2 secretion.

FIG. 9 Effect of different FGF2 concentrations on SK-N-MC cells on the quotient of red fluorescent nuclei and green fluorescence caused by apoptotic cells.

FIG. 10 Effect of secreted FGF2 concentrations on SK-N-MC cells co-incubated with HeLa cells secreting FGF2 inducible by doxycycline on the quotient of red fluorescent nuclei and green fluorescence caused by apoptotic cells.

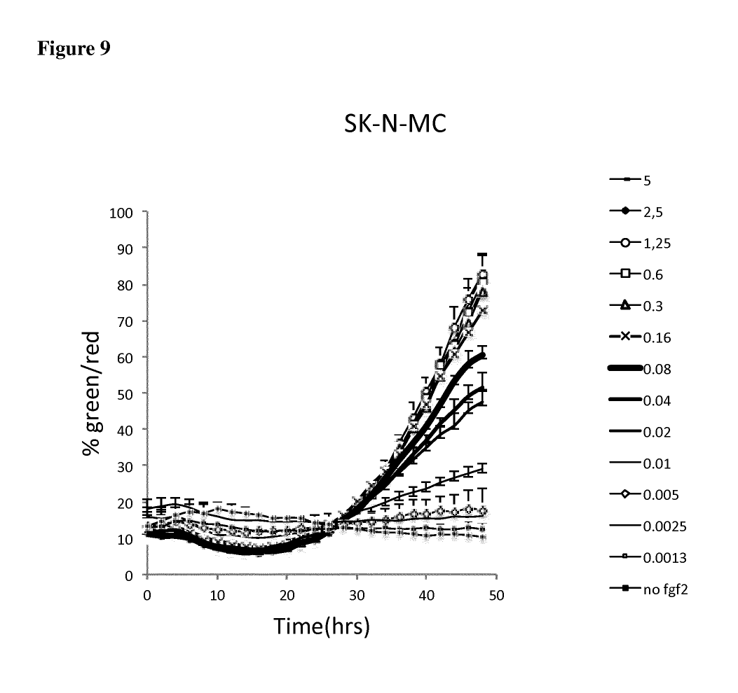

FIG. 9 Titration of recombinant FGF2 and its effect on target cells

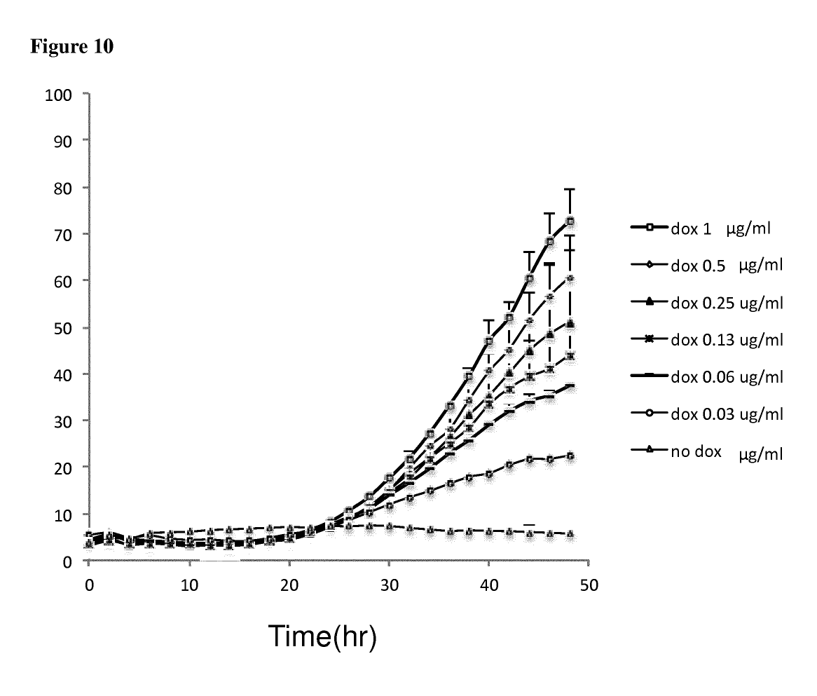

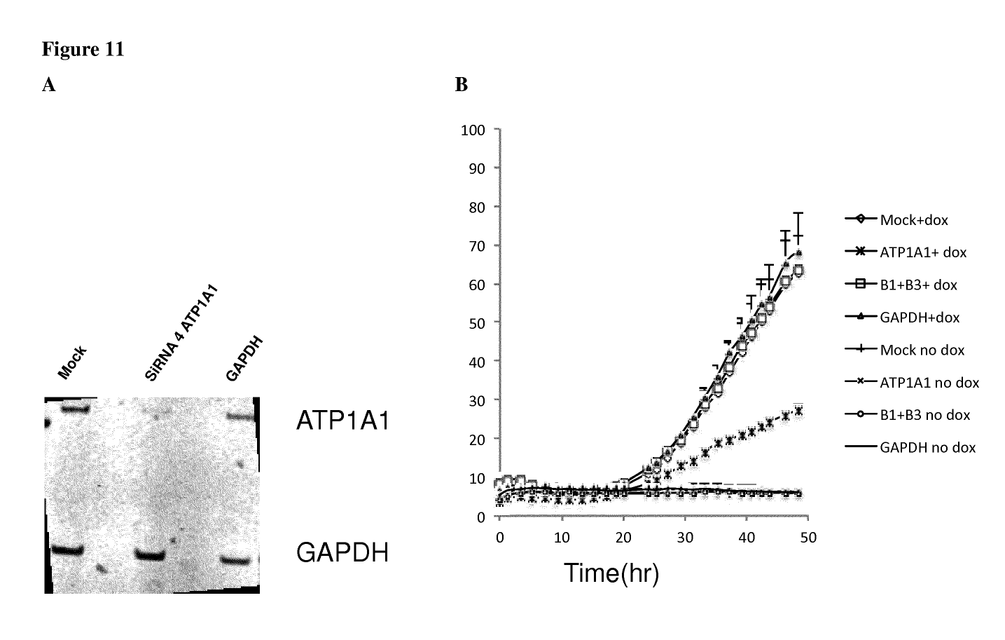

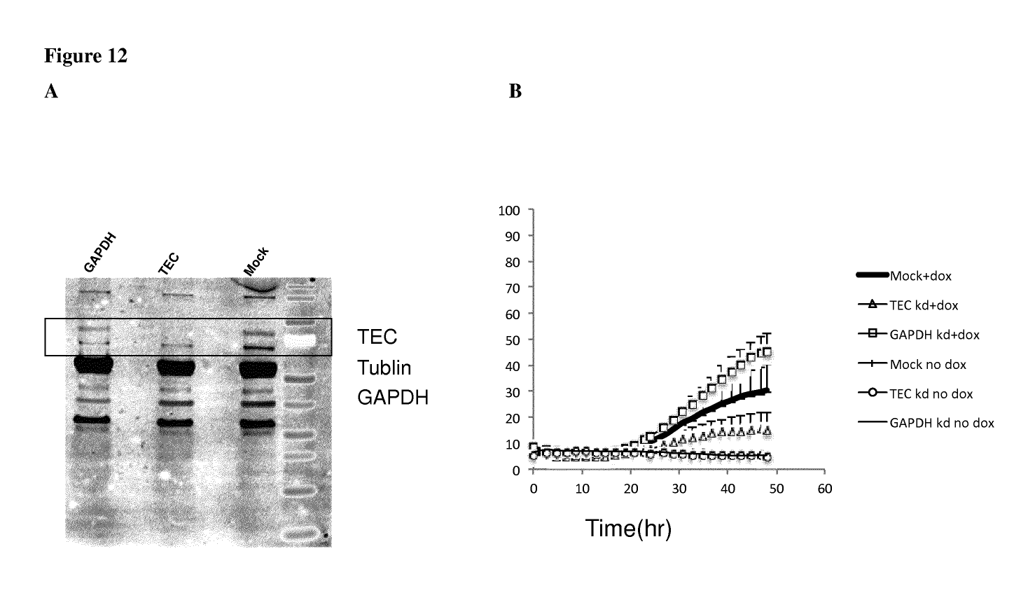

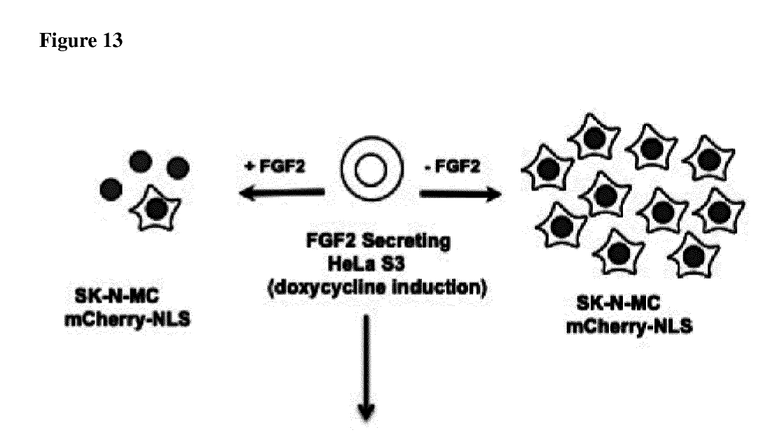

FIG. 10 Dependence of ratio of apoptotic cells to living cells from induced FGF2 concentration secreted by coincubated HeLa cells

FIG. 11 Dependence of quotient of apoptotic cells to living cells from knock-down of ATP1A1 and the presence of doxycycline and GAPDH: A—Expression of ATP1A1; B—Effects of knock down

FIG. 12 Dependence of ratio of apoptotic cells to living cells from knock-down of Tec-kinase and the presence of doxycycline: A—Expression of Tec-kinase; B—Effects of knock down.

FIG. 13 Schematic depiction of quantitative assay

### DETAILED DESCRIPTION OF THE INVENTION

The present disclosure provides a method for the identification of compounds modulating the secretion of FGF2 or FGF2 signaling by employing experiments distinguishing the absence versus presence of recombinant FGF2.

Modulation of FGF2 secretion according to the present disclosure refers to the activation or suppression of FGF2 signaling. In this context, modulation of FGF2 secretion may also refer to influencing the nature or the character of FGF2 and its secretion, either by affecting the level of FGF2 intra- or extracellular, which is still developing or maturing or by blocking secretion of FGF2 into the EM or by altering its molecular signaling capacity by blocking its binding to receptors. The disclosed assay might also reveal compounds that block downstream signaling in target cells, like SK-N-MC cells. However, the disclosed methods provides besides quantification of FGF2 that is present a tool allowing qualitative analysis. IF target cells will be unaffected by co-incubation with other cells or compounds, the addition of recombinant FGF2 allows to determine whether the signalling pathway of the target cells is affected. If recombinant FGF2 is able to induce target cells to proliferation it becomes obvious that the before observed lack of proliferation oft the target cells is not related to the signalling pathways in the target cells. In case that the addition of recombinant FGF2 will not be able to induce proliferation of the target cells, the compound or co-incubated cells will affect the cellular machinery of the target cells or the recombinant FGF2 directly.

According to the present disclosure, target cells represent a cell line, which is sensitive to the presence of FGF2, caused by a shut-down of cell proliferation and subsequent cell death. FGF2 secreting cells represent a cell line, which is able to secret FGF2 and control cells represent a cell line, which is insensitive to the presence of FGF2.

A reporter within the meaning of the present invention shall be understood as a gene encoding a protein, which can be used as a reporter like a fluorescent protein. The target cells may have been stably transfected with such a gene of a first reporter, so that it will be constitutively expressed. The control cells may have been stably transfected with such a gene of a different second reporter, so that it will be constitutively expressed.

A compound library according to the present disclosure refers to a collection of chemicals, further called compounds, and their related information. Compounds are selected from the group comprising peptides, proteins, carbohydrates, antibodies, lipids, micelles, vesicles, synthetic molecules and polymers.

A “solid phase” to which compounds to be investigated can be covalently or non-covalently attached refers to, but is not restricted to, a column, a matrix, beads, glass including modified or functionalized glass, synthetic membranes, silica or silica-based materials including silicon and modified silicon, plastics (comprising polypropylene, polyethylene, polystyrene and copolymers of styrene and other materials, acrylics, polybutylene, polyurethanes etc.), nylon or nitrocellulose, resins, polysaccharides, carbon as well as inorganic glasses, metals, nanoparticles, and plastics. Thus, microtiter plates are also within the scope of a solid phase according to the present disclosure.

The method according to the invention may be used very efficiently for the identification of cell growth disorders, wherein cell growth disorders comprise tumour or cancer associated diseases like leukemia. The tumour disease can be a disease selected from the group comprising tumours of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs comprising non-parvicellular bronchial carcinomas, parvicel-lular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarial carcinoma, tumors of the uterine tube (Tuba Faloppii), tumors of the abdominal cavity, mammary carcinomas, tumors of the endo-crine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocu-lar melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, morbus Hodgkin, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancy such as Kaposi sarcoma, AIDS-associated lymphomas, AIDS-associated lymphomas of the central nervous system, AIDS-associated morbus Hodgkin and AIDS-associated anogenital tumors, transplantation-related malignancy, metastasized tumors comprising brain metastases, lung metastases, liver metastases, bone me-tastases, pleural and pericardial metastases, and malignant ascites.

The invention will be described by experiments and figures. It is obvious for a person ordinary skilled in the art that the invention is not limited to the disclosed embodiments.

SK-N-MC cells, a human neuroepithilioma cell line (ATCC HTB-10), were stably transduced with a construct carrying the red fluorescent protein mCherry fused to a nuclear localization signal (mCherry-NLS). The cell line used in the shown experiments is derived from a single clone that was isolated by FACS resulting in homogenous red fluorescence in the nuclei of this cell population. Using an Essen Biosciences Incucyte Zoom LED microscope, the proliferation of these cells can be monitored in a quantitative manner for an almost unlimited period of time.

Since SK-N-MC cells are known to respond to FGF2 by a shut-down of cell proliferation, they can be used to sense FGF2 in the extracellular space. For example, as a proof of principle, this property of FGF2 can be demonstrated by challenging SK-N-MC cells with recombinant FGF2 (25 ng/ml). Furthermore, we can make use of this phenomenon to monitor FGF2 secretion from a second cell line based on co-cultivation of SK-N-MC cells with for example Hela S3 cells (ATCC CCL-2.2) expressing FGF2 in a doxycycline-dependent manner.

For experiments using recombinant FGF2, SK-N-MC cells expressing mCherry-NLS were cultivated in 96 well plates (Corning) at a starting density of 5000 cells per well. Cells were grown in Minimum Essential Medium Eagle alpha modified (α-MEM, Sigma) supplemented with 10% FCS, Penicillin (100 U/ml) and Streptomycin (100 μg/ml). Recombinant FGF2 (18 kDa form with N-terminal His-tag) was added at a final concentration of 25 ng/ml in α-MEM.

For co-culturing experiments, HeLa S3 cells expressing FGF2 were seeded at 1000 cells per well in 96 well plates (Corning) followed by the addition of doxycycline (1 μg/ml) to induce FGF2 expression and secretion. Following 48 hours of cultivation, SK-N-MC cells constitutively expressing mCherry-NLS were added at a density of 5000 cells per well. Cells were grown in α-MEM in an appropriate incubator at 5% CO₂ and 37° C.

To specifically monitor cell proliferation of SK-N-MC cells labelled with mCherry-NLS, live imaging was performed using an Essen Biosciences Incucyte Zoom Instrument along with a statistical analysis of three technical replicates for each experimental condition. Images were acquired every 2 hours with four images taken per well for each time point. This system allows for an absolute quantification of fluorescent nuclei and, therefore, a direct measurement of cell proliferation over time.

Since SK-N-MC cells will become apoptatic in the presence of FGF2, a caspase 3/7 assay was used to detect DNA of apoptotic cell with a green fluorescent dye. Theoretically, FGF2 should result in a decrease of red nuclei of living cells and an increase of green colour caused by an increasing amount of apoptotic cells among red SK-N-MC cells.

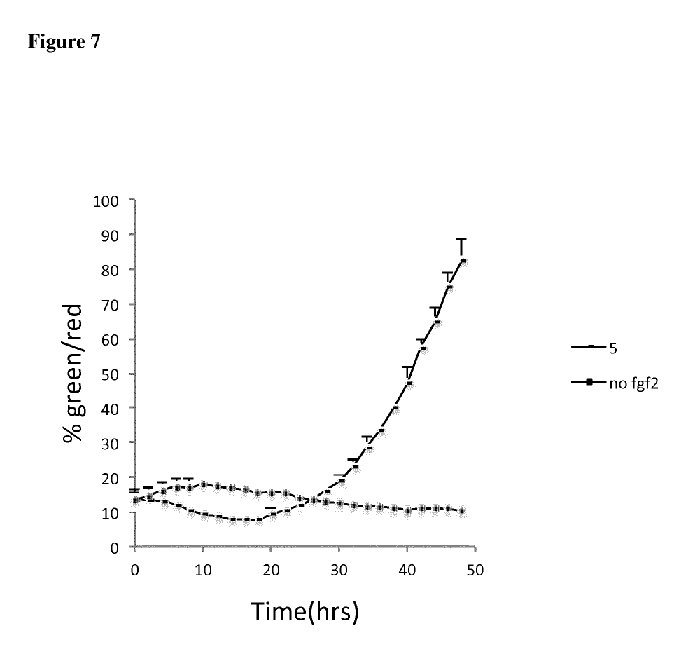

By calculating the ratio of green versus red fluorescence it should be possible to get a more precise result or relation between FGF2 concentration change in the proliferation of the target cells. Corresponding results are shown in FIGS. 7 to 10. FGF2 was titrated and added directly or FGF2 secretion was induced by adding doxycycline to HeLa cells comprising an doxycycline inducible FGF2 gene.

Surprisingly the calculation of the quotient of red fluorescent nuclei and green fluorescence caused by apoptotic cells resulted in more robust results reflecting the effect of the respective concentration of FGF2 on the proliferation of the target cells.

A key approach in the identification of molecular components involved in FGF2 secretion has been a genome-wide RNAi screen that led to the identification of Tec kinase as a regulatory component of FGF2 secretion (Nickel et al., 2011, Traffic 12, 799-805, 32; Ebert, et al., 2010), Traffic 11, 813-82). In addition to Tec kinase, this screen also revealed ATP1A1 as a gene product whose down-regulation causes a substantial drop in FGF2 secretion efficiency.

Knock-downs of ATP1A1 and Tec-kinase were done in the FGF2 expressing cell line followed by quantifying FGF2 secretion using the disclosed assay employing SK-N-MC cells.

The instant invention discloses an assay employing a target cell line sensitive to FGF2 secretion that can be used to quantify the concentration of FGF2 and allows to determine whether inhibition of FGF2 secretion is caused in the FGF2 donor cells or the target cell line. Thus, it is not only possible to identify FGF2 secretion and/or signalling effecting molecules, but also to get information about their mode of action. Using the method utilising one or two fluorescent parameters allows for a precise determination of presence and—if a calibration took place prior to measuring for FGF2—concentration of FGF2.

### DETAILED DESCRIPTION OF THE FIGURES

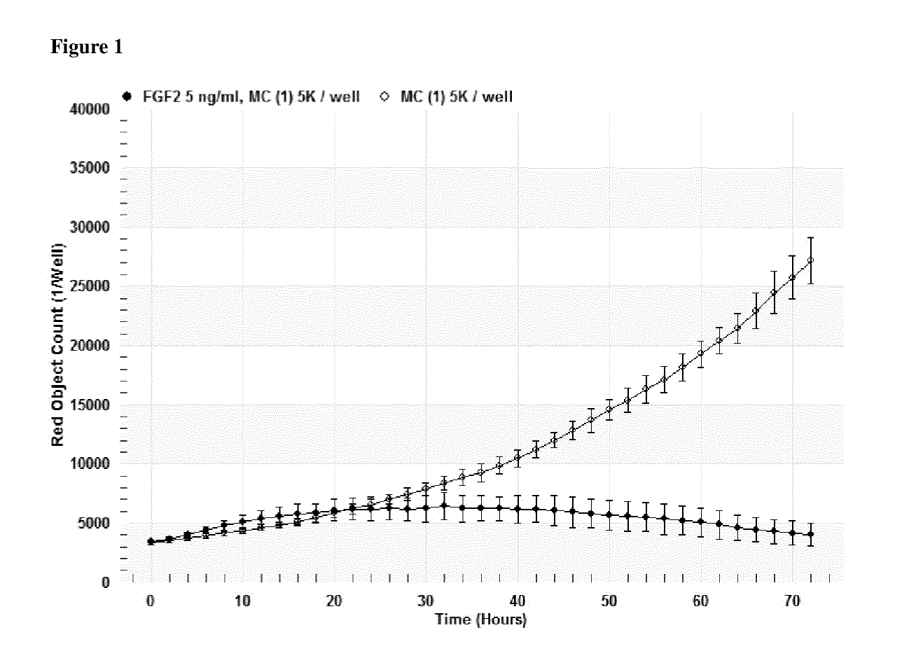

FIG. 1 shows a target cell line, which is sensitive to the presence of FGF2. The cells express constitutively a red fluorescent protein coupled to a nuclear localisation signal, so that the nuclei of living cells appear to be red. The presence of recombinant FGF2 (5 ng/ml) results in a reduction of cells. The target cells are neuroblastoma cells (Neuroblastoma SK-N-MC) that were stably transfected with a red fluorescent protein. In the absence of recombinant FGF2 the cells proliferate normally.

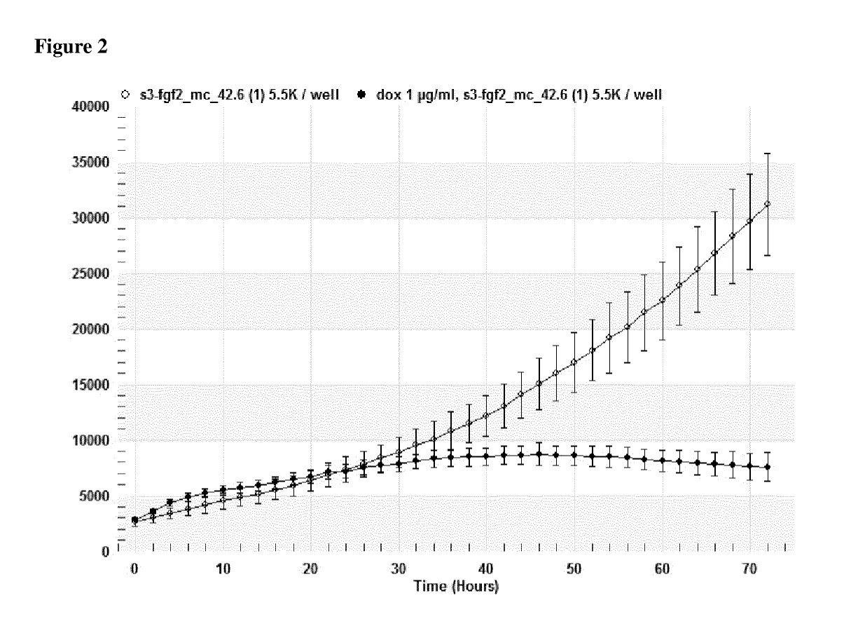

FIG. 2 shows the results of co-incubating target cells expressing constitutively a RED fluorescent reporter with HeLa cells secreting FGF2 inducible by doxycycline. The upper line in FIG. 1 shows co-incubation of the cells without inducing FGF2 secretion by doxycycline. After induction of FGF2 secretion by addition of doxycycline a massive reduction of the number of target cells can be observed.

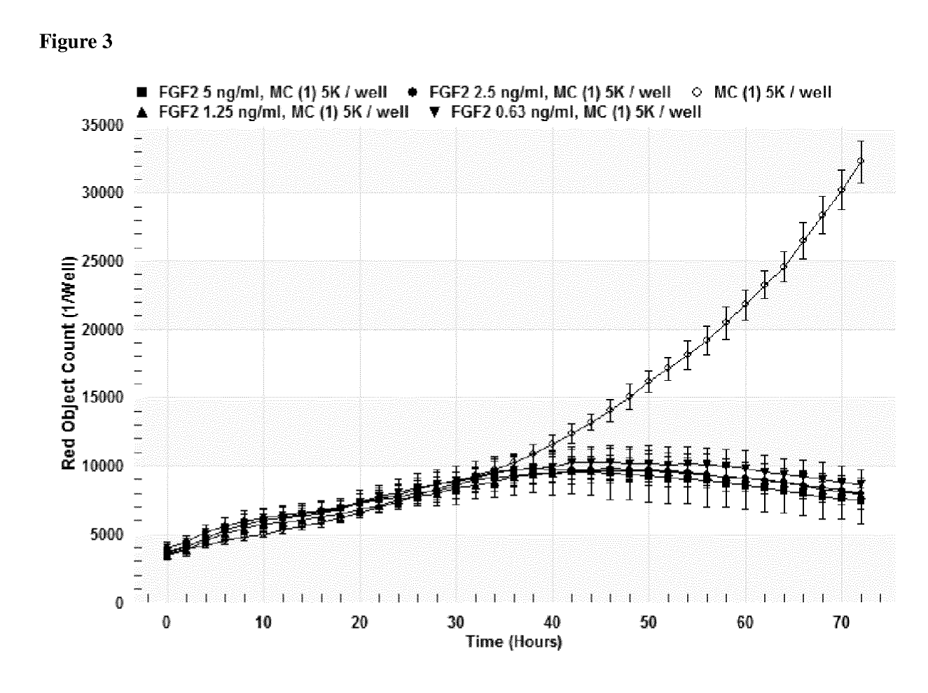

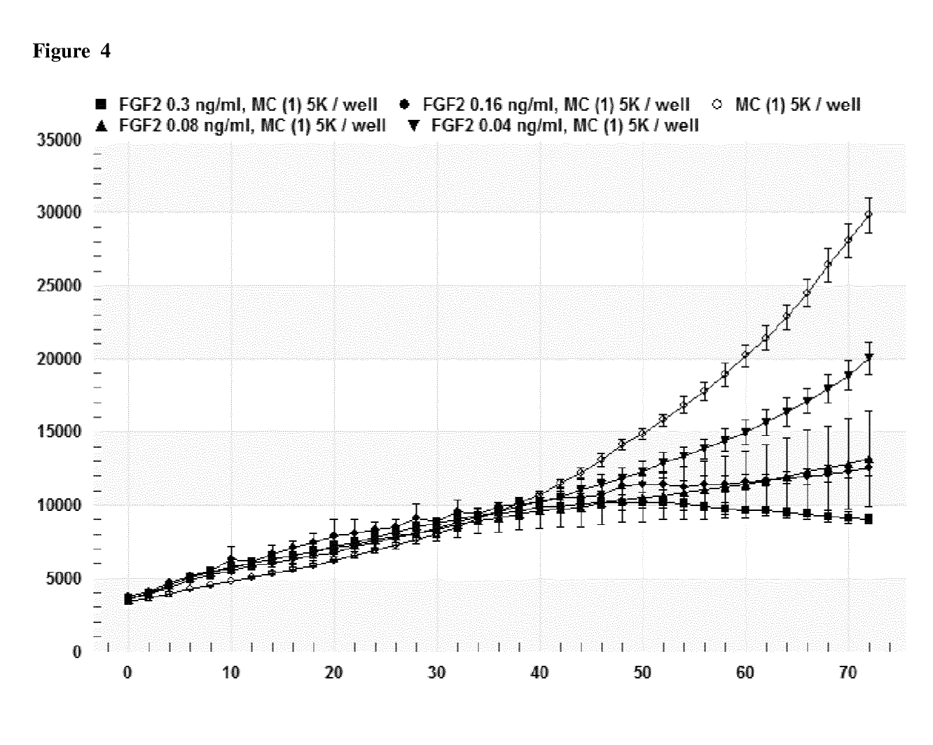

FIGS. 3 and 4 show effects of different concentrations of FGF2 on the cell growth of the target cell line sensitive to the presence of FGF2. It can be observed that the growth curves depend on the respective concentration of FGF2, so that less FGF2 results in a better growth of the cells.

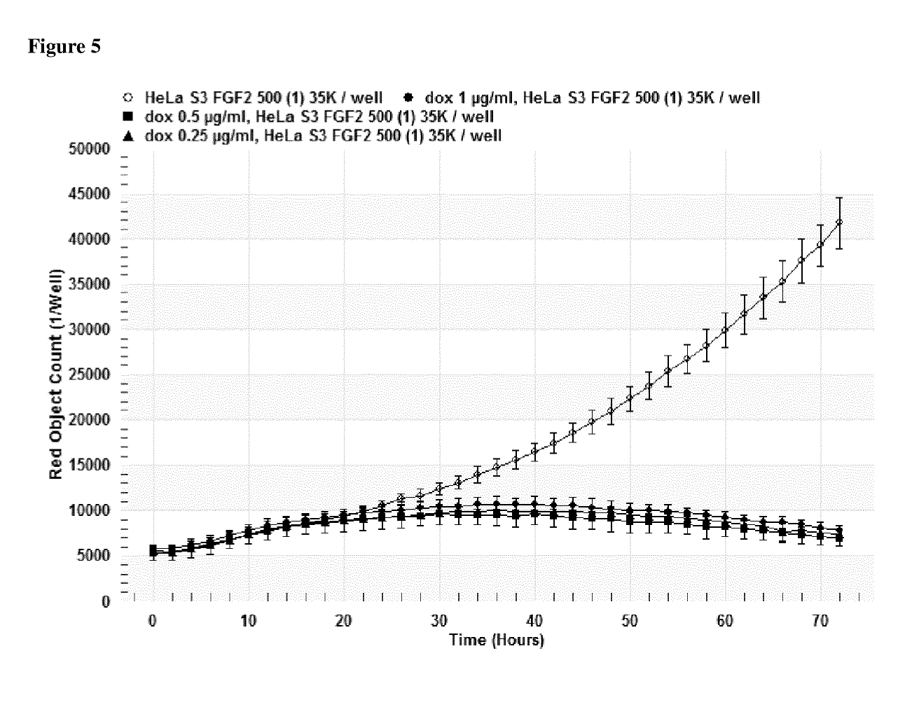

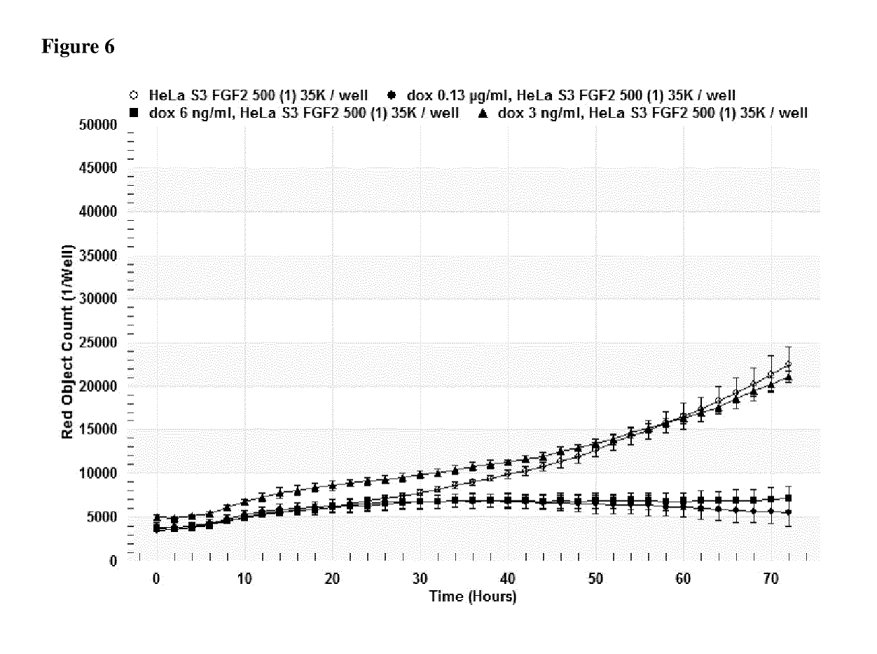

FIGS. 5 and 6 show effects of adding different amounts of doxycycline to the medium in order to induce FGF2 secretion by co-incubated HeLa cells. Again, the amount of induced FGF2 secretion by the addition of doxycycline (dox) effect the proliferation of the target cells in a way that the more FGF2 is secreted the less proliferation of target cells can be observed.

The results in FIGS. 3-6 show that the proliferation of the target cells is dose dependent with respect to the presence of FGF2 even if FGF2 secretion is the effect of adding different amounts of on inducer for FGF2 secretion. Thus, the assay seems to be surprisingly suitable to prepare calibrated growth curves so that a quantification of FGF2 concentrations will be possible.

FIG. 7 shows the quotient of red fluorescent nuclei and green fluorescence caused by apoptotic cells in the absence and presence of FGF2. It has to be noted that the output of the results reverses the previously shown curves, because the induction of apoptosis by FGF2 will result in an increase of the quotient of green to red fluorescence.

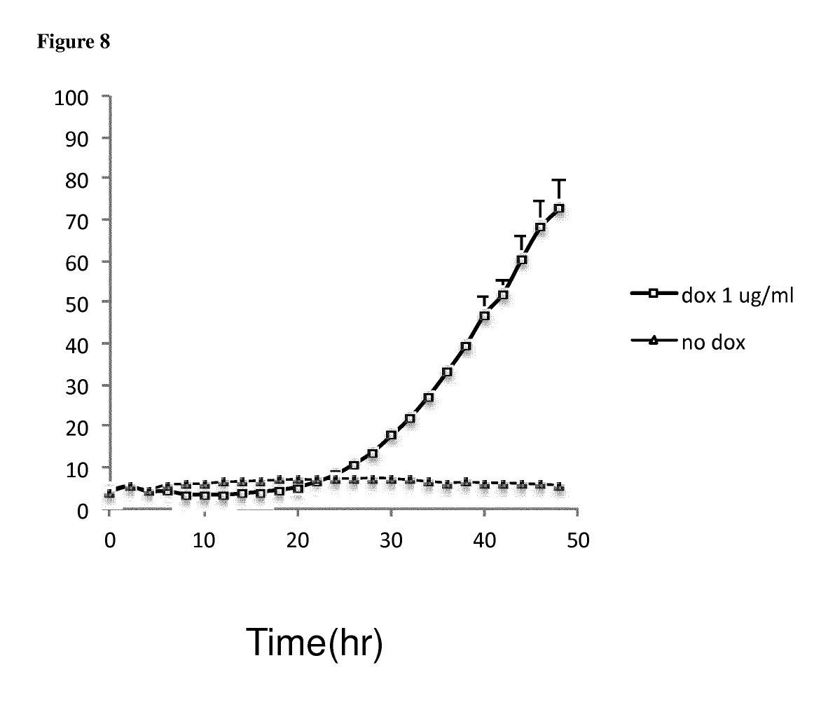

FIG. 8 shows the quotient of red fluorescent nuclei and green fluorescence caused by apoptotic cells in the absence and presence of doxycycline induced FGF2 secretion. The quotient increases if doxycycline is added in a concentration of 1 μg/ml due to the increase of apoptotic cells.

FIG. 9 shows the effect of different FGF2 concentrations on SK-N-MC cells on the quotient of red fluorescent nuclei and green fluorescence caused by apoptotic cells. The quotient is dose dependent and reflects the dose dependency more precisely than using only red fluorescence of nuclei of living target cells.

FIG. 10 shows the effect of secreted FGF2 concentrations on SK-N-MC cells co-incubated with HeLa cells secreting FGF2 inducible by doxycycline on the quotient of red fluorescent nuclei and green fluorescence caused by apoptotic cells. Different doxycycline concentrations were added which is reflected in the quotient of living and apoptotic cells.

FIG. 11 shows in part A the knock down of ATP1A1 expression ond in part B the effect of the knock down and GAPDH (control knock-down) and/or doxycycline addition. It was surprisingly possible to detect the knock-down of ATP1A1 by using the assay calculating the quotient of red and green fluorescence caused by living cells (red) and apoptotic cells (green).

FIG. 12 shows in part A the knock down of Tec-kinase expression ond in part B the effect of the knock down and GAPDH (control knock-down) and/or doxycycline addition. It was surprisingly possible to detect the knock-down of Tec-kinase as well by using the assay calculating the quotient of red and green fluorescence caused by living cells (red) and apoptotic cells (green).

1. A method for the identification of the presence of FGF2, the method comprising the use of target cells sensitive to the presence of FGF2 by obstructing proliferation, wherein the target cells constitutively express a first reporter in the nucleus of living target cells.

2. The method of claim 1, wherein the first reporter is a first fluorescent protein.

3. The method of claim 1, wherein a second reporter is used for detecting apoptotic cells.

4. The method of claim 3, wherein the second reporter is a second fluorescent protein.

5. The method of claim 4, wherein the absorption spectrum of the first and second fluorescent protein differ.

6. The method of claim 1, wherein the fluorescence of first and/or second reporter is determined.

7. The method of claim 6, wherein the quotient of the fluorescence of the first and second reporter is calculated.

8. The method of claim 1, wherein the target cells are co-incubated with cells or a liquid to detect the secretion or presence of FGF2.

9. The method of claim 8, wherein the target cells are co-incubated with FGF2 secreting cells

10. The method of claim 9, wherein the FGF2 secretion in the FGF2 secreting cells is inducible.

11. The method of claim 9, wherein the FGF2 secreting cells are coincubated with at least one compound selected from the group comprising peptides, proteins, nucleic acids, carbohydrates, antibodies, lipids, micelles, vesicles, synthetic molecules and polymers.

12. The method of claim 2, wherein the number of living target cells is monitored by determining fluorescence of the first reporter.

13. The method of claim 9, wherein the FGF2 secreting cells are selected from the group comprising tumor cells, endothelial cells, muscle cells, neural cells and fibroblasts.

14. The method of claim 1, wherein additionally control cells are used, which are constitutively expressing a third reporter, wherein the control cells are insensitive to the presence of FGF2.

15. The method of claim 1, wherein the target cells are genetically modified cells derived from a neuroblastoma.

16. The method of claim 1, wherein the target cells are SK-N-MC cells.

17. The method of claim 1, wherein at least one compound library is used for co-incubation with the target cells, with compounds being bound to at least one of metal particles, nanoparticles, or a solid phase and the compounds being selected from the group comprising peptides, proteins, carbohydrates, antibodies, lipids, micelles, vesicles, synthetic or biological molecules and polymers.

18. The method of claim 17, wherein at least one compound library is used for co-incubation with the target cells, wherein the compounds of the library are in solution.

19. The method of claim 1, wherein the target cells are cultivated in multiwell plates with each well comprising different compounds to be tested for their ability to modulate FGF2 secretion and/or signalling.

20. The method of claim 1, wherein FGF2 secreting cells are separated from target cells by cell sorting, such as fluorescence activated cell sorting or magnetic cell sorting, and re-cultured for further approaches.

21. (canceled)

22. A kit for the identification of FGF2 modulating compounds, the kit comprising target cells constitutively expressing a first reporter in the nucleus having a first fluorescence, wherein the target cells are sensitive to the presence of FGF2.

23. The kit of claim 22, wherein comprising a second reporter having a second fluorescence with a different absorption spectrum from the first fluorescence of the first reporter, wherein the second reporter detects apoptotic cells.

24. The kit of claim 22, wherein the kit comprises control cells constitutively expressing a third reporter having a third fluorescence different from the first and second fluorescence of first and second reporter, wherein the control cells are insensitive to the presence of FGF2.

25. The kit of claim 22, wherein the kit comprises recombinant FGF2.

26. The kit of claim 22, wherein the kit comprises FGF2 secreting cells, wherein wih FGF2 secretion is inducible.

27. The present invention relates to a method for the identification of compounds modulating the biological activity of FGF2, the method comprising the use of target cells sensitive to the presence of FGF2 by obstructing proliferation, wherein the target cells constitutively express a first reporter in the nucleus of living target cells.
